(19) United States
(12) Patent Application Publication
(10) Pub. No.: US 2024/0374862 A1
(43) Pub. Date: Nov. 14, 2024

(54) Pranayam Machine

(71) Applicants: Daxesh Patel, Mansa (IN); Hetal Patel, Mansa (IN); Joy Patel, Mansa (IN); Kalp Patel, Ahmedabad (IN)

(72) Inventors: Daxesh Patel, Mansa (IN); Hetal Patel, Mansa (IN); Sahil Kalal, Dungarpur (IN); Pritish Prajapati, Gandhinagar (IN)

(21) Appl. No.: 18/577,685

(22) PCT Filed: Apr. 9, 2022
(86) PCT No.: PCT/IN2022/050348

§ 371 Date: Jan. 8, 2024

(30) Foreign Application Priority Data
Aug. 25, 2021 (IN) 202121038548

(51) Int. Cl. A61M 21/02; A61F 11/12; A61M 21/00

(52) U.S. Cl. CPC A61M 21/02; A61F 11/12; A61M 2021/0022; A61M 2021/0088; A61M 2205/103; A61M 2205/8206; A61M 2209/088; A61M 2210/0618

(57) ABSTRACT

A Pranayam Machine disclosed in present invention is a device that facilitates the performance of certain yogic breathing practices without the use of hands, it provides two mechanisms, mechanisms 1 and mechanism 2 in which parts that are common in both the mechanisms, are Servo motor (3), motor to fork coupling (4), Belts (6) and Ear plugs (7). Design of Forehead mounting (2) and Fork (5) differs depending on mechanism 1 and mechanism 2. This Forehead mounting (2) precisely fits on one's Head (1) through adjusting Belts (6). The major difference between Forehead mounting (2) in both the mechanisms, is that in mechanism 1, Servo motor (3) is mounted perpendicular to the surface and the Fork (5) is mounted perpendicular to the Servo motor (3) shaft axis, whereas in mechanism 2 Servo motor (3) is mounted parallel to the surface and the Fork (5) is mounted along the axis of the Servo motor (3).

### FIELD OF INVENTION

The present invention generally relates to health equipment and more particularly to a device that facilitates the performance of certain yogic breathing practices such as Anulom Vilom, Nadi Suddhi, Bhramari Pranayama and others without the use of hands, while achieving the desired outcome.

### BACKGROUND OF INVENTION

Pranayama is the art and science of yogic breathing techniques, and these techniques will reliably produce the benefits of physical well-being, lightness of heart, clarity of mind, inner and outer health and fulfillment of purpose, intention, and direction. Breathing is one of the most important and intricate activities we engage in. Cellular respiration, the source of all our physical energy and expression in the world, is dependent on a constant flow of oxygen being delivered to each cell and carbon dioxide being taken away.

A comprehensive system of such breathing techniques is found, for example, in yoga. While yoga addresses an all-embracing family of spiritual practices, into which physical exercises (asanas) and breathing exercises are performed. These ancient techniques or similar exercises have entered into modern practices of physiotherapy and relaxation exercises. So the number of people practicing breathing exercises will significantly exceed those doing yoga. Positive effects are documented for these techniques in terms of a basic sense of well-being, but it is also known that these techniques support stress relaxation, can help improve sleep and problem solving capabilities as well as the cardiopulmonary performance. Usually, all of these techniques should be learned from an instructor and practiced (at home) on a regular basis, preferably daily. It is common to practice at home self-directly or with the use of an audio tape or CD, where an instructor guides through the different exercises. The exercises are to be done in a relaxed position, preferably sitting or lying, and in a concentrated state of mind, which means with a minimum of distraction. Yoga is reported to reduce stress and anxiety, improves autonomic functions by triggering neuro hormonal mechanisms by the suppression of sympathetic activity, and even, now-a-days, several reports suggest yoga is beneficial for physical health of cancer patients and other critical conditions too. Such global recognition of yoga also testifies to India's growing cultural influence.

Anulom Vilom, Nadi Suddhi, Bhramari Pranayama and others have turned out to be very beneficial to one who practices them regularly. Moreover, performing them on a daily basis, for about 30-60 mins twice a day, have proven that they can not only prevent many diseases and keep oneself fit, but have also cured many diseases. Major problems faced by people who want to practice this pranayama is that, due to continuous involvement of one's hand, sometimes unable practitioners attain adequate time required for these pranayama, as it leads to pain in hands, elbow joint and fingers. To overcome these issues many individuals, reduce time involvement in this pranayama to about 5 minutes, and the drawback of such hacks is that it reduces the effect of such exercise and expected results are not gained. Sometimes many individuals get bored after 5 minutes of anulom vilom, nadi suddhi, bhramari pranayam and others, as it requires continuous physical involvement.

Yogic practices have been followed since ancient times because of their positive impact on human health and well-being. The certain Yogic breathing practices eg. Anulom vilom pranayam are performed with the assistance of one hand fingers used to block or unblock nostrils while breathing. This means at least one hand is thus engaged, and one may be able to not do other tasks that require the use of either both hands, or the occupied hand. In addition, one has to remain focused to keep track of the breathing patterns and count the number of cycles specific to the technique being practiced. Thus most other tasks that require focus and attention cannot be performed, since incorrect patterns or number of cycles during yogic breathing may not be very effective. In light of the foregoing discussion, there is a need for a device (and a manufacturing method thereof) that is suitable for facilitating the performance of certain yogic breathing practices without the use of hands, and which can be programmed or automated to perform specific patterns and cycles of certain yogic breathing techniques, while still achieving the desired outcome.

An invention disclosed in patent application number WO2010140158A1 discloses a device, for performing certain ancient yogic breathing practices of Pranayam without involving the use of hands, is controlled by a programmable controlling unit. The device is integrated onto the frame of a supporting base, worn on the nose, and uses battery-operated artificial fingers to block or unblock nostrils in programmable patterns, cycles and durations. This facilitates a precise and hands-free routine of alternate nostril breathing or single nostril breathing, in the most precise style, following techniques of ancient yogic Pranayama. The sensors of the device sense/measure the force and flow of the breath into and out of each nostril, and can automate and synchronise the artificial fingers to the normal breathing pattern of the user.

An invention disclosed in patent application number WO2008139380A2 discloses a system and method for guiding breathing exercises, a method of processing a sensor signal related to breathing and a use thereof. The invention describes a system that allows a guided breathing exercise, where the intensity, frequency and duration of exercises can be adapted to the actual performance of the exercising user. This is obtained by monitoring throughout the exercise the process of in-and exhalation and bated breath. This information is used to adapt the actual timing of each of these phases to the desired rhythm of the specific exercise. Also the intensity of inhalation and exhalation can be monitored and adjusted. The user is instructed with the adjusted exercise, preferably by feedback in audible, visual or tactile manner.

An invention disclosed in patent application number KR20200127521A discloses a headphone combined breathing meditation induction device capable of measuring brain waves, comprising a body mounted on a user's face and a control box electrically coupled to the body and providing a driving signal to the body. The main body includes first and second opening and closing means for opening and closing the nostril of the user's nose, first and second driving means respectively driving the first and second opening and closing means, and a mounting for mounting the body on the face. In the breathing meditation induction device combined with headphones capable of measuring brain waves, characterized in that the control box is configured by including a means, and further comprising a memory card holder for inserting a memory card, the inner side of the headphone installation unit. A pair of (electroencephalogram) EEG measurement electrodes capable of measuring EEG, which is a kind of bio-signal, is installed, and one side includes a sensing means for EEG measurement consisting of one grounding electrode, and for coupling to a personal computer or smartphone. It is characterized in that it includes a USB interface, is connected to a personal computer or a smartphone, operates according to a measurement request signal of the user using a personal computer or smartphone, and transmits the EEG signal sensed by the personal computer or smartphone. A breathing meditation induction device combined with headphones that senses an EEG signal for transmitting.

One of the major problems faced by people practicing Anulom Vilom, are physical problems such as pain in the tennis elbow. To overcome such problems, the present novel invention is an automatic anulom vilom machine which is powered by a Li-ion battery. This device consists of a forehead mounting, a fork, and an embedded system. For actuating the fork, a servo motor is used. The present device provides one size which fits all. As this device is to be mounted on the forehead, so by adjusting its mounting on the forehead, it can fit the majority of people. This is accomplished by using three strap elastic models. Even long term usage of the present novel machine won't lead to rashes on any part of the face. Not only anulom vilom pranayama, but also nadi suddhi, bhramari pranayama and others can also be performed through this device for a longer period without the usage of hands and fingers.

### OBJECTIVE OF THE INVENTION

Principal objective of the present invention is to provide a device that facilitates the performance of certain yogic breathing practices such as Anulom Vilom, Nadi Suddhi, Bhramari Pranayama and others without the use of hands, while achieving the desired outcome.
Still another object of the present invention is to provide a device that is easy to install on a supporting base.
Still another object of the present invention is to provide a device that can be used to perform certain yogic breathing practices using alternate nostrils, both nostrils or a single nostril.
Still another object of the present invention is to provide a device that synchronizes the device with the patterns of human breathing.
Still another object of the present invention is to provide a device that can be programmed for desired need.
Another objective of the present invention is to provide a portable and handy device that can be used and transferred anywhere according to the user's need.
Still another object of the present invention is to provide a device that is easy to use.

### SUMMARY

To overcome the problem stated in the background, the present invention provides a perfect combination of highly efficient mechanisms as well as accurate design which is powered by an adequate processing unit. The present yoga device consists of different sets of parts such as; forehead mounting, fork and belt. Whereas, the processing unit includes a microcontroller, nob, ON/OFF switch and battery. With the help of a nob that is present in the processing unit one can easily control frequency of anulom vilom and nadisuddhi pranayama, depending on one's breathing cycles. Moreover, one who is using the present yoga device can perform pranayama for 30-60 minutes, without any side effects on one's body, thus overcoming the problem stated above.

### LIST OF DRAWINGS

FIG. 1: Circuit diagram

FIG. 2: Flowchart of embedded system

FIG. 3: Mechanism 1 forehead mounting

FIG. 4: Front view of mechanism 1 forehead mounting

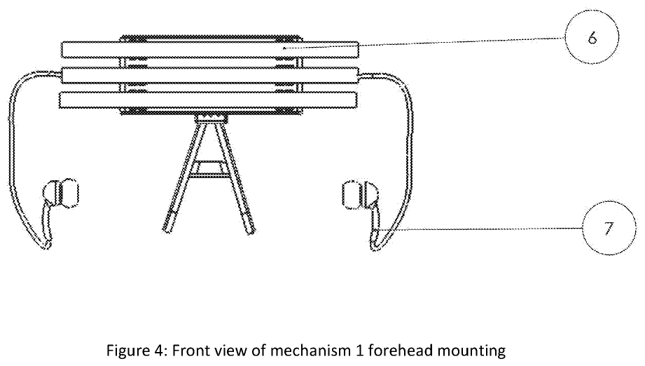

FIG. 5: Side view of mechanism 1 forehead mounting

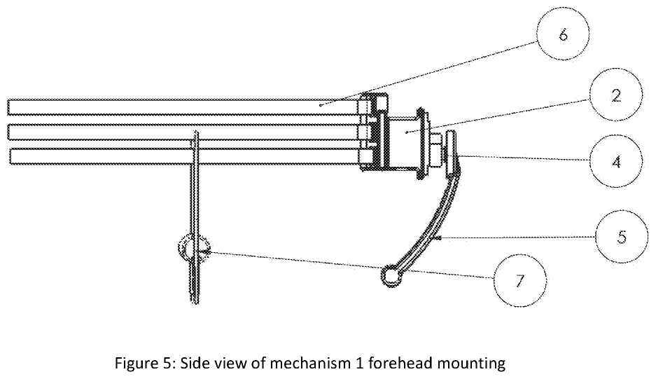

FIG. 6: Mechanism 2 forehead mounting

FIG. 7: Front view of mechanism 2 forehead mounting

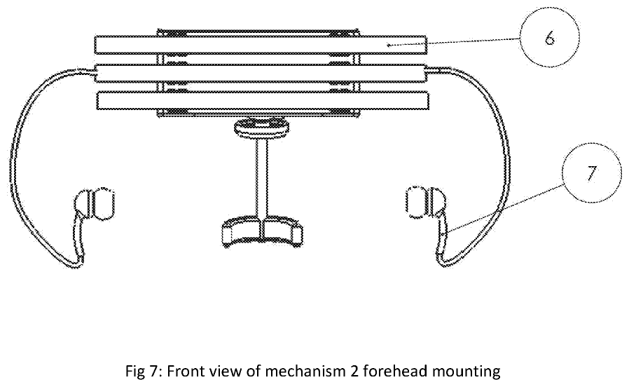

FIG. 8: Side view of mechanism 2 forehead mounting

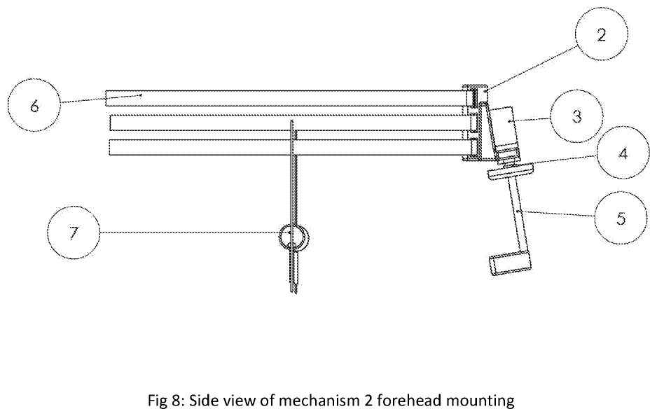

### LIST OF COMPONENTS

1. Head
2. Forehead mounting
3. Servo motor
4. Fork coupling
5. Fork
6. Belts
7. Ear plugs
8. Microcontroller
9. Battery
10. On/Off Switch
11. Regulated Power Supply
12. Potentiometer/Knob

### DETAILED DESCRIPTION OF DRAWINGS AND INVENTION

The present novel invention Pranayam Machine has Head (1), Forehead mounting (2), Servo motor (3), Fork coupling (4), Fork (5), Belts (6), Ear plugs (7), Microcontroller (8), Battery (9), On/Off Switch (10), Regulated Power Supply (11), and Potentiometer/Knob (12).

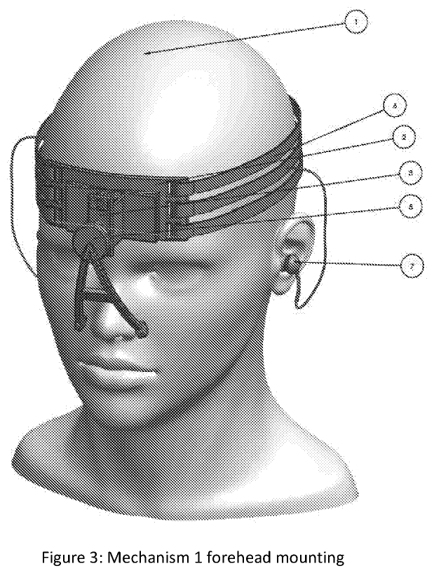

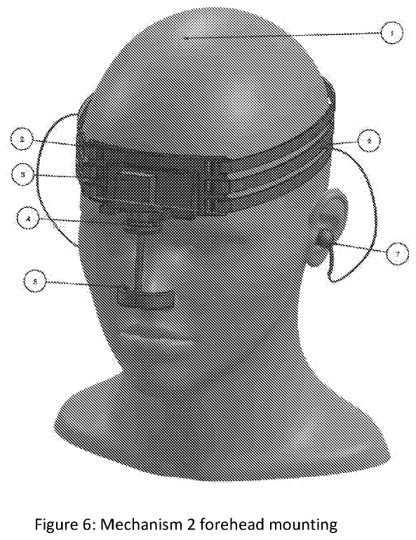

The present invention provides mechanisms 1 and mechanism 2 as shown in FIGS. 3 and 6 respectively, in which Forehead mounting (2) is supposed to be placed on one's forehead. Design of Forehead mounting (2) differs depending on mechanism 1 and mechanism 2. This Forehead mounting (2) precisely fits on one's Head (1) through adjusting Belts (6). In the present invention, there are three Belts (6) connected with Forehead mounting (2), the Belt (6) can be changed through different kinds of available materials having flexibility so as such a structure gives a better fixture with Head (1). The Belt (6) can be of one strap or more straps. Moreover, Ear plugs (7) are also connected with Forehead mounting (2). Servo motor (3) is connected with Forehead mounting (2) through bolts and/or fixtures, threads for these bolts are provided on Forehead mounting (2). Position of these threads differ based on which mechanism is considered out of mechanism 1 or mechanism 2. The Servo motor (3) is mounted to Fork coupling (4) through its shaft. Servo motor (3) is connected with an embedded system. Furthermore, this Fork coupling (4) is connected with a Fork (5). Design of Fork (5) also differs depending on which mechanism is considered. Thus, parts of which are common in both the mechanism, mechanism 1 and mechanism 2 are Servo motor (3), motor to fork coupling (4), Belts (6) and Ear plugs (7). And parts which differ in both the mechanisms, mechanism 1 and mechanism 2 are Forehead mounting (2) and Fork (5).

Embedded system of the present invention majorly consists of Servo motor (3), Microcontroller (8), Battery (9), ON/OFF switch (10), Regulated power supply (11), and Potentiometer/Knob (12). In the present invention Servo motor (3) is a 180-degree servo motor that has a torque range of 1.8-2.0 kg·cm, which is enough to press the surface of the nose as well as to avoid any sort of injury, similarly any other motor can also be used to achieve the same. Fork coupling (4) is used to transfer torque efficiently from Servo motor (3) to Fork (5). In the present invention Forehead mounting (2) is a part on which Servo motor (3) is attached with four bolts/screws, it can also be fixed in any other means too. The major difference between Forehead mounting (2) in both the mechanisms is that, in mechanism 1 Servo motor (3) is mounted perpendicular to the surface, whereas in mechanism 2 Servo motor (3) is mounted parallel to the surface.

In the Mechanism 1, the Fork (5) is mounted perpendicular to the Servo motor (3) shaft axis. Whereas, in the mechanism 2, the Fork (5) is mounted along the axis of the Servo motor (3). Sizes do vary according to the length of the human nose and soft silicon is used on the nose touching part.

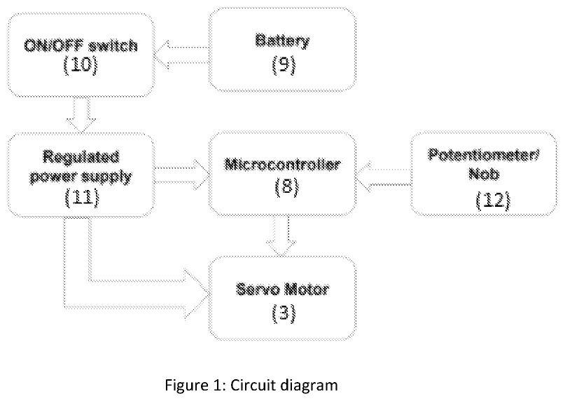

As mentioned in FIG. 1 circuit diagram, it describes the embedded system of the present novel invention where Microcontroller (8) is controlling all the processing of the system. The Microcontroller (8) is fetched with 5V of regulated power supply form Regulated Power Supply (11). Furthermore, Microcontroller (8) receives analog signal from Potentiometer/Knob (12), which is a variable resistor. This Potentiometer/Knob (12) helps in varying the frequency of Servo motor (3). Servo motor (3) receives signal from Microcontroller (8), which receives form Potentiometer/Knob (12). Thus, through this embedded system the frequency of Fork (5) varies. This whole embedded system has been fetched with >6V Battery (6) power supply. The present novel invention uses two Li-ion cell Battery (9), between Battery (9) and Regulated Power Supply (11), there is an ON/OFF Switch (10) through which one can ON or OFF the present novel yoga device.

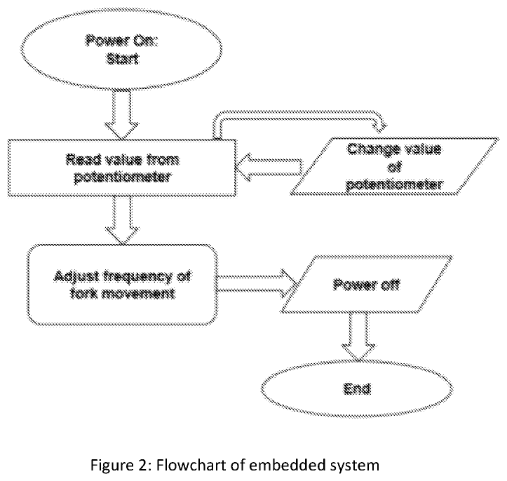

As mentioned in FIG. 2 flowchart of embedded system, describes the functioning of this embedded system. First the said system gets powered ON through ON/off Switch (10). Then Microcontroller (8) continuously reads analog values generated by Potentiometer/Knob (12). Thus, based on the varying value received through Potentiometer/Knob (12) frequency of Fork (5) is changed. And after switching OFF the ON/off Switch (5), the whole embedded system is powered OFF and no further command is executed by Microcontroller (8).

We claim:

1. A Pranayam Machine, which facilitates the performance of yogic breathing practices like as Anulom Vilom, Nadi Suddhi, Bhramari Pranayama and others without the use of fingers and hands, while achieving the desired outcome using alternate nostrils, both nostrils or a single nostril and synchronizes with the patterns of human breathing, wherein it is comprising of:
Head (1), Forehead mounting (2), Servo motor (3), Fork coupling (4), Fork (5), Belts (6), Ear plugs (7), Microcontroller (8), Battery (9), On/Off Switch (10), Regulated Power Supply (11), and Potentiometer/Knob (12);
Wherein the Forehead mounting (2) fits on one's Head (1) through the adjusting Belts (6), the Ear plugs (7) and the Servo motor (3) are also connected with Forehead mounting (2), the said Servo motor (3) is mounted to the Fork coupling (4) through its shaft and the said Fork coupling (4) is connected with the Fork (5), the nose touching part of the said Fork (5) is made of silicon;
Wherein the Microcontroller (8) receives analog signal from the Potentiometer/Knob (12), by which the frequency of Servo motor (3) varies, accordingly the frequency of Fork (5) also varies;
Wherein the ON/OFF Switch (10) is fitted between Battery (9) and Regulated Power Supply (11).

2. The Pranayam Machine as claimed in claim 1, wherein in the mechanism 1, the Servo motor (3) is mounted perpendicular to the surface.

3. The Pranayam Machine as claimed in claim 1, wherein in the mechanism 2, the Servo motor (3) is mounted parallel to the surface.

4. The Pranayam Machine as claimed in claim 1, wherein in the mechanism 1, the Fork (5) is mounted perpendicular to the Servo motor (3) shaft axis.

5. The Pranayam Machine as claimed in claim 1, wherein in the mechanism 2, the Fork (5) is mounted along the axis of the Servo motor (3).

6. The Pranayam Machine as claimed in claim 1, wherein the Servo motor (3) is having at least 20-degree movement with a torque capacity of at least 1 kg·cm.

7. The Pranayam Machine as claimed in claim 1, wherein the Potentiometer/Knob (12) is used to change the frequency of the Fork (5) according to user's need.

8. The Pranayam Machine as claimed in claim 1, wherein the device is powered by at least 3v battery (6).

9. The Pranayam Machine as claimed in claim 1, wherein Belts (6) connected with Forehead mounting (2) is made of flexible material, so as to give a proper fixture with the Head (1).
